(19) United States
(12) Patent Application Publication
(10) Pub. No.: US 2019/0167992 A1
(43) Pub. Date: Jun. 6, 2019

(54) APPARATUS, METHOD AND MACHINE-READABLE MEDIUM OF PROVIDING ELECTRICAL STIMULATION THERAPY TO TREAT PAIN

(71) Applicant: WEHEAL BIOTECHNOLOGY COMPANY, KAOHSIUNG CITY (TW)

(72) Inventor: Chi-Jung Tai, Kaohsiung City (TW)

(21) Appl. No.: 15/832,437

(22) Filed: Dec. 5, 2017

(51) Int. Cl. A61N 1/36; A61B 5/00; A61B 5/053; A61N 1/02

(52) U.S. Cl. CPC A61N 1/36021; A61N 1/36031; A61N 1/025; A61B 5/0531; A61B 5/4824

(57) ABSTRACT

An apparatus, a method and machine-readable medium for providing electrical stimulation therapy to treat pain are provided. The method may comprise receiving a first information, mapping the first information to a first parameter, generating a first notification signal in accordance with the first parameter, and transmitting the first notification signal. The method further comprises receiving a second information subsequent to the transmission of the first notification signal, mapping the second information to a second parameter, generating a second notification signal in accordance with the second parameter, and transmitting the second notification signal.

### FIELD

The present disclosure relates generally to an apparatus, a method and a machine-readable medium for providing electrical stimulation therapy to treat pain.

### BACKGROUND

An electrical stimulators may be used to deliver electrical stimulation therapy or signals to patients to treat a variety of symptoms or conditions, e.g. chronic pain, tremor, Parkinson's disease, epilepsy, urinary or fecal incontinence, sexual dysfunction, obesity, or gastroparesis. For example, a stimulator may deliver neurostimulation therapy in the form of electrical pulses. A stimulator may deliver neurostimulation therapy via one or more leads or pads that include electrodes located proximate to target locations associated with a target area of a patient.

A patient has to go to a clinic or hospital where a clinician selects values for a number of programmable parameters to define the electrical stimulation therapy to be delivered by the stimulator to the patient. However, the visit to a clinic or a hospital may in itself pose a risk of infection due to the physical proximity of the patient to other patients at the medical facility (at the waiting rooms for example). In addition, traveling to and from the medical facilities is time consuming, and inconvenient especially when feeling ill.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 is a schematic diagram of an electronic device in accordance with some embodiments of the subject application.

FIG. 2 is a schematic diagram of an electronic device in accordance with some embodiments of the subject application.

FIG. 3 is a schematic diagram of an apparatus in accordance with some embodiments of the subject application.

FIG. 4 is a block diagram illustrating a system in accordance with some embodiments of the subject application.

FIG. 5 illustrates a method for performing a method for providing electrical stimulation therapy to treat pain.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

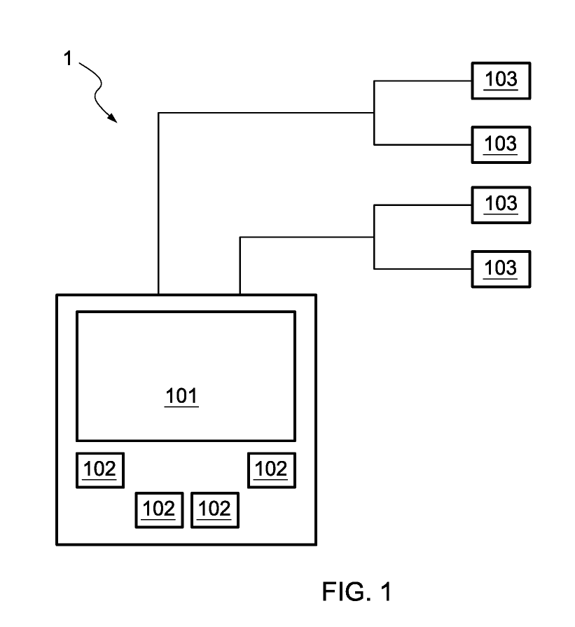

FIG. 1 illustrates a schematic diagram of an electronic device 1 in accordance with some embodiments of the subject application. The electronic device 1 includes a display 101, an input interface 102 and patches 103. FIG. 1 shows an external structure or a profile of the electronic device 1.

The display 101 may include but is not limited to e.g. a screen, a LED display, a touch screen, a touch panel, or the like.

The input interface 102 may include a number of touch pads, buttons or human interface device (HID) for inputting or selecting information (or data). The input interface 102 may serve as function selection input, e.g. start, stop or pause, to control the electronic device 1.

The patches 103 or pads 103 may be attached to or disposed on a target area (or painful area, e.g. neck, check, arm, leg, foot, etc.) of a body. The patches 103 or pads 103 may be used to facilitate measurement of skin-impedances of the body, to facilitate a nerve conduction velocity (NCV) test or a nerve conduction study (NCS) of the body, or to facilitate other applications.

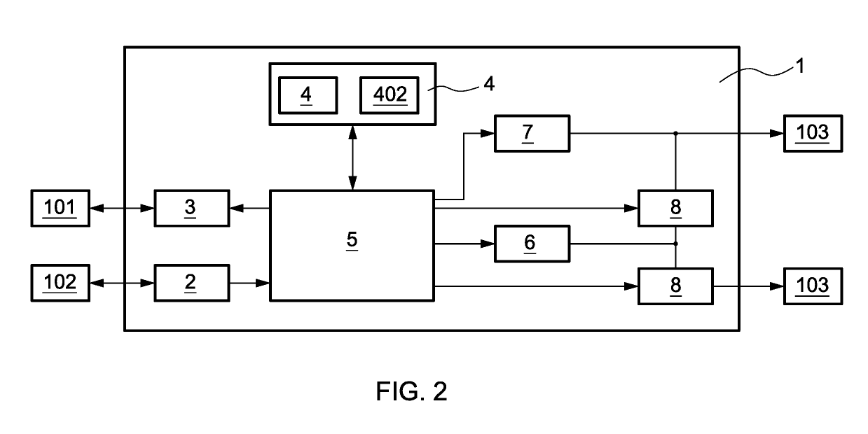

FIG. 2 illustrates a schematic diagram of an electronic device in accordance with some embodiments of the subject application. FIG. 2 illustrates internal structure of the electronic device 1.

The internal structure of the electronic device 1 includes an input module 2, a display module 3, a communication module 4, a controller 5, a skin-impedance/NCV/NCS determining module 6, a pulse generator 7 and switch modules 8.

The input module 2 may transform signals from the input interface 102 (as shown in FIG. 1) into electronic signals. The input module 2 is electrically connected to the input interface 102. Information or data (e.g. user descriptions) can be input by the input interface 102.

The display module 3 is electrically connected to the display 101 (as shown in FIG. 1). The display module 3 may include a circuitry or a driver to control or drive the display 101.

The controller 5 may include but is not limited to, for example, a controller, a processor, a micro control unit (MCU), a central processing unit (CPU), etc. The controller 5 is electrically connected to the input module 2. The controller 5 is electrically connected to the display module 3. The controller 5 receives signals from the input module 2. The controller 5 may control the display module 3 to display images on the display 101 (as shown in FIG. 1).

The communication module 4 may include but is not limited to, for example, a wireless communication module, a Wi-Fi module, a Bluetooth module, a wired communication module (e.g. an ethernet communication module), etc. The communication module 4 may include a receiver 401 for receiving signals. The communication module 4 may include a transmitter 402 for transmitting signals. The communication module 4 is electrically connected to the controller 5.

The skin-impedance/NCV/NCS determining module 6 is electrically connected to the controller 5. The skin-impedance/NCV/NCS determining module 6 is electrically connected to the patches 103. The skin-impedance/NCV/NCS determining module 6 receives signals from the patches 103 to determine a skin-impedance value. The controller 5 and the skin-impedance/NCV/NCS determining module 6 use the signals from the patches 103 to determine a skin-impedance value. The skin-impedance/NCV/NCS determining module 6 helps the controller 5 using the signals from the patches 103 to determine a skin-impedance value.

The pulse generator 7 is electrically connected to the controller 5. The pulse generator 7 is electrically connected to the patches 103. The pulse generator 7 may generate signals or stimulation signals. A signal or stimulation signal generated by the pulse generator 7 may include but is not limited to, for example, a square waveform, a triangular waveform, a sinusoidal waveform, a ramp waveform. The pulse generator 7 may generate signals or stimulation signals having different waveforms. The pulse generator 7 may generate signals or stimulation signals of different frequencies. The pulse generator 7 may generate signals or stimulation signals having different amplitudes. The pulse generator 7 may receive signals, commands, or instructions from the controller 5.

The controller 5 may instruct the pulse generator 7 to generate signals or stimulation signals. The generated signals are output to the user via the patches 103. The skin-impedance/NCV/NCS determining module 6 receives signal(s) from a body via the patches 103 to determine skin-impedance or nerve conduction velocity. The controller 5 and the skin-impedance/NCV/NCS determining module 6 use signal(s) from a body via the patches 103 to determine skin-impedance or nerve conduction velocity. The skin-impedance/NCV/NCS determining module 6 helps the controller 5 using signal(s) from a body via the patches 103 to determine skin-impedance or nerve conduction velocity.

Each of the switch modules 8 is electrically connected to the controller 5. Each of the switch modules 8 is electrically connected to one another. Each of the switch modules 8 is electrically connected to each other. Each of the switch modules 8 is electrically connected to the skin-impedance/NCV/NCS determining module 6. Each of the switch modules 8 is electrically connected to the patches 103. Each of the switch modules 8 is electrically connected to a pair of patches 103. When measuring the skin-impedance or nerve conduction velocity, the controller 5 instructs the switch module 8 to switch the skin-impedance/NCV/NCS determining module 6 on and switch the pulse generator 7 off. When providing electrical stimulation therapy, the controller 5 instructs the switch module 8 to switch the pulse generator 7 on and switch the skin-impedance/NCV/NCS determining module 6 off.

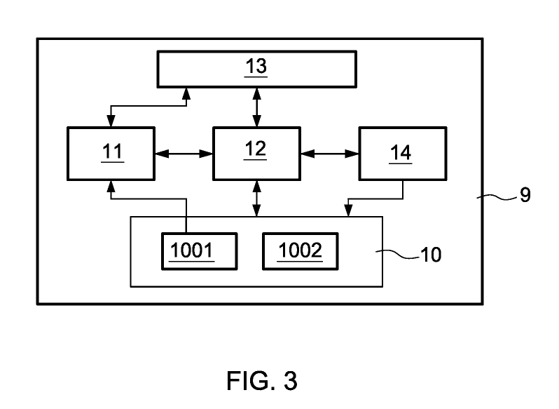

FIG. 3 is a schematic diagram of an apparatus in accordance with some embodiments of the subject application. Referring to FIG. 3, the apparatus 9 has a communication module 10, a mapping module 11, a controller 12, a storage unit/database 13, and a signal generator 14.

The apparatus 9 may include but is not limited to, for example, a computer, a server, etc. The apparatus 9 may communicate with the electronic device 1 as shown FIG. 1. The apparatus 9 may communicate with the electronic device 1 as shown FIG. 2. The apparatus 9 may communicate with the electronic device 1 as shown FIG. 1 via another electronic device (e.g. mobile/cellular phone, laptop, etc.) with communication module(s).

The communication module 10 is same or similar to the communication module 4 as described and illustrated with reference to FIG. 2. The communication module 10 is different from the communication module 4 as described and illustrated with reference to FIG. 2. The communication module 10 includes a receiver 1001 for receiving information or signals. The communication module 10 may include a transmitter 1002 for transmitting information, instructions or signals.

The controller 12 is same or similar to the controller 5 as described and illustrated with reference to FIG. 2. The controller 12 is different from the controller 5 as described and illustrated with reference to FIG. 2, for example, the controller 12 may include cloud computing process which has relatively great performance over the controller 5 as described and illustrated with reference to FIG. 2. The controller 12 is electrically connected to the communication module 10.

The mapping module 11 is electrically connected to the controller 12. The mapping module 11 is electrically connected to the communication module 10. The mapping module 11 maps information received form the receiver 1001 to parameter(s) in the storage unit/database 13.

The storage unit/database 13 is electrically connected to the controller 12. The storage unit/database 13 is electrically connected to the mapping module 11. The mapped parameter may be stored in the storage unit/database 13.

The signal generator 14 is electrically connected to the controller 12. The signal generator 14 is electrically connected to the communication module 10. The controller 12 controls the signal generator 14 to generate a notification signal in accordance with the mapped parameter. The controller 12 controls the signal generator 14 to generate a notification signal in accordance with the mapped parameter in the storage unit/database 13.

The notification signal is then sent to the transmitter 1002 of the communication 10. The transmitter 1002 transmits the notification signal. The controller 12 receives the mapped parameter from the mapping module 11 and process to generate instruction(s) to control the signal generator 14 to generate a notification signal. The controller 12 may obtain the mapped parameter in the storage unit/database 13 and process to generate instruction(s) to control the signal generator 14 to generate a notification signal.

Although not illustrated in FIG. 3, it is contemplated that the mapping module 11 may be incorporated or integrated into the controller 12. Although not illustrated in FIG. 3, it is contemplated that the signal generator 14 may be incorporated or integrated into the controller 12.

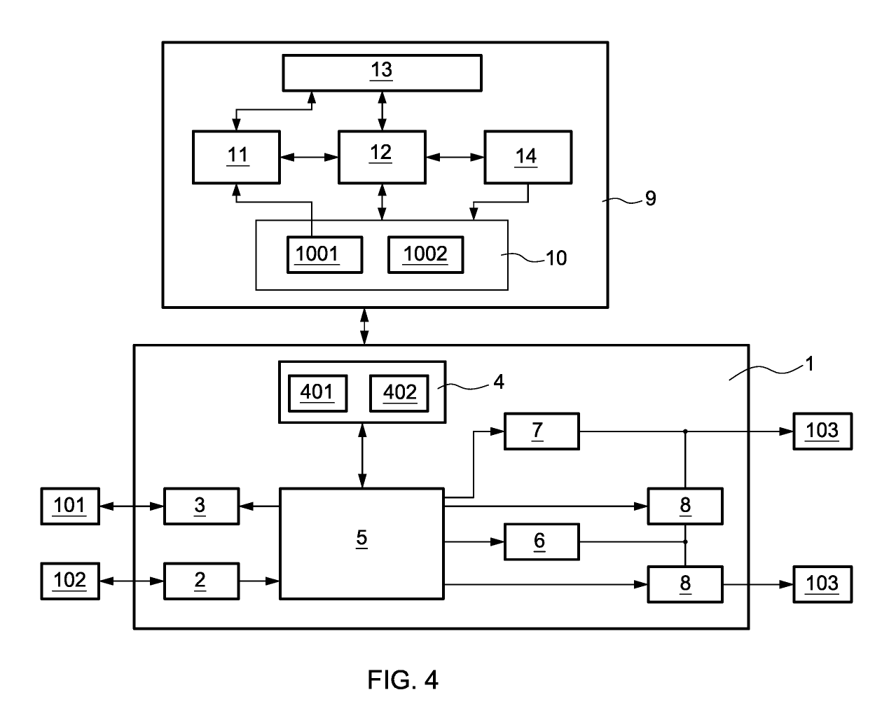

FIG. 4 is a block diagram illustrating a system in accordance with some embodiments of the subject application. Referring to FIG. 4, the system may include the electronic device 1 as described and illustrated with reference to FIG. 2 and the apparatus 9 as described and illustrated with reference to FIG. 3.

The input module 2 receives a first information from the input interface 102 and sends it to the controller 5. The first information may be received by the apparatus 9 with the help of the communication module 4 and the communication module 10. The first parameter may include one or more of an area of interest, a frequency of a stimulation signal, a waveform of a stimulation signal, a period/time duration of a stimulation signal, or a magnitude of a stimulation signal, etc.

The mapping module 11 maps the first information to a first parameter stored in the storage unit/database 13. The mapping module 11 maps the first information to a first parameter. The first parameter is sent to a controller 12. The first parameter is stored in the storage unit/database 13. The controller 12 may generate instruction(s) based on the data (e.g. the first parameter or the mapped parameter) stored in the storage unit/database 13. The controller 12 may control the signal generator 14 to generate a first notification signal in accordance with the first parameter.

The first notification signal is transmitted to the electronic device 1. The controller 5 receives the first notification signal and generates instruction(s) to control the pulse generator 7 to output signals to the patches 103. The controller 5 receives the first notification signal and generates instructions to control the skin-impedance/NCV/NCS determining module 6 to determine a first skin-impedance. The first skin-impedance may be sent to the apparatus 9 and be stored in the storage unit/database 13.

The controller 5 may send the instructions to the pulse generator 7. The pulse generator 7 generates a first stimulation signal in accordance with the instructions of the controller 5.

The first information includes a first value indicating a pain level. The first information includes a painful area of the user. The first information includes user's description. The first information includes a first skin-impedance of the user.

The user may input second information or descriptions after receiving the first stimulation signal. The input module 2 receives the second information from the input interface 102 and sends the second information to the controller 5. The second information includes a second value indicating a pain level. The second information includes a second skin-impedance of the user.

The second information may be received by the apparatus 9 with the help of the communication module 4 and the communication module 10.

The mapping module 11 maps the second information to a second parameter. The mapping module 11 maps the second information to a second parameter in the storage unit/database 13. The second parameter may comprise a frequency of a stimulation signal, a waveform of a stimulation signal, a duration of a stimulation signal, etc.

The second parameter is sent to the controller 12. The second parameter is stored in the storage unit/database 13. The controller 12 may generate instruction(s) based on the data (e.g. the second parameter or the mapped parameter) stored in the storage unit/database 13. The controller 12 may control the signal generator 14 to generate a second notification signal in accordance with the second parameter.

The second notification signal is transmitted to the electronic device 1. The controller 5 receives the second notification signal and generates instruction(s) to control the pulse generator 7 to output signals to the patches 103. The controller 5 receives the second notification signal and generates instructions to control the skin-impedance/NCV/NCS determining module 6 to determine a second skin-impedance. The second skin-impedance may be sent to the apparatus 9 and be stored in the storage unit/database 13.

The controller 5 may send the instructions to the pulse generator 7. The pulse generator 7 generates a second stimulation signal in accordance with the instructions of the controller 5.

The switch modules 8 can decide whether to transmit the second stimulation signal and/or measure the second skin-impedance of the user. When the second stimulation signal is sent to the patches 103 or pads 103 and output to the user, the user may control the magnitude of the second stimulation signal output from the electronic device 1 himself/herself. Descriptions can be input through the input 102 to the electronic device 1 after the second stimulation signal is output to the user.

The apparatus 9 can compare the first and second values. If the second value is different from the first value, the second parameter is different from the first parameter. The second notification signal thus is thus different from the first notification signal. The second stimulation signal is also different from the first stimulation signal. In some embodiments, the first parameter may contain one or more of the following variables: an area of interest, a frequency of an electrical signal, a waveform of the electrical signal, a period/time duration of the electrical signal, a magnitude of the electrical signal, and etc.

For example, if five kinds of the above-mentioned variables are used, there will be 120 possible permutations for the first parameter. A first stimulation signal based on the first parameter is output to the user. The user may input second information (which may include descriptions) after receiving the first stimulation signal.

If the user feels that the pain level he/she feels increases, the user inputs a second value larger than the first value. The apparatus 9 may adopt one of the other 119 permutations (except the previous determined permutation) as the second parameter and generates the second notification signal in accordance with the second parameter.

If the user feels that the pain level he/she feels decreased, the user inputs a second value equal to the first value, and thus the second parameter is the same as the first parameter. The apparatus 9 may adopt the same parameter and generates the same notification signal. The second notification signal is thus identical to the first notification signal. The second stimulation signal is also the same as the first stimulation signal.

If the user feels that the pain level he/she feels decreases relatively great, the user inputs a second value less than the first value. The apparatus 9 may change some of the variables and keep other variables of the first parameter unchanged as the second parameter, and generates the second notification signal in accordance with the second parameter.

The first information and second information may include the first and second skin-impedances, respectively. The first and second skin-impedances are stored in the storage unit/database 13. If the second skin-impedance is different from the first impedance, the pain level that the user feels may be different. Thus the first and second information input by the user may be different. As a result, the second parameter is different from the first parameter.

The storage unit/database 13 may be set up or updated, e.g. the products and the kind of medical services that the users need, may be built in accordance with principles of medical science and/or Chinese medical Twelve Main Meridians and stored in the storage unit/database 13.

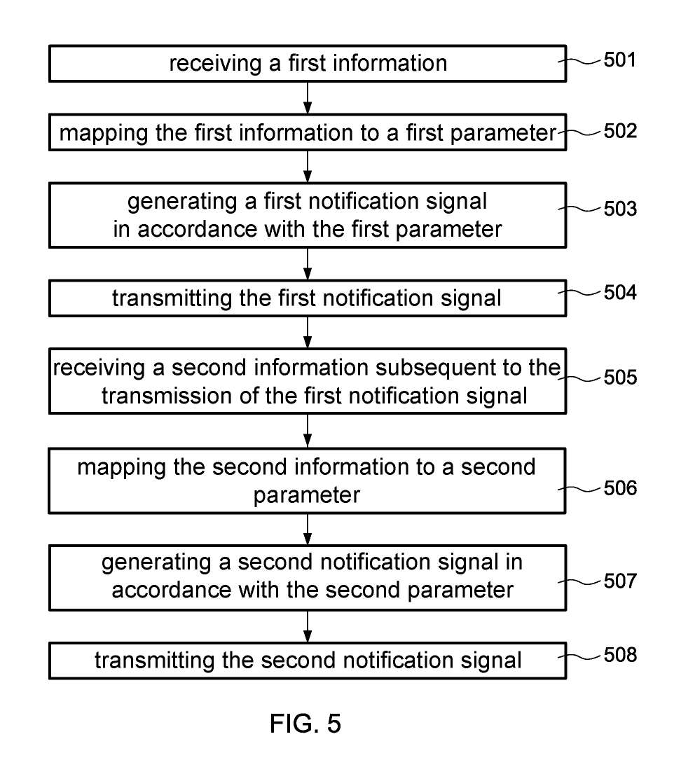

FIG. 5 illustrates a method for performing a method for providing electrical stimulation therapy to treat pain. In 501, a first information is received. In 502, the first information is mapped to a first parameter. In 503, a first notification signal is generated in accordance with the first parameter. In 504, the first notification signal is transmitted. In 505, a second information is received subsequent to the transmission of the first notification signal. In 506, the second information is mapped to a second parameter. In 507, a second notification signal is generated in accordance with the second parameter. In 508, the second notification signal is transmitted.

The first information includes a first value indicating a pain level. The first information also comprises a painful area. The first information may comprise the user's description. The first information further comprises a first impedance.

The first parameter includes one or more of an area of interest, a frequency of an electrical signal, a waveform of the electrical signal, a period/time duration of the electrical signal or a magnitude of the electrical signal.

The second information includes a second value indicating a pain level. The second information also comprises a second impedance.

The second parameter is different from the first parameter while the second value is different from the first value. The second parameter is the same as the first parameter while the second value is equal to the first value. The second parameter is different from the first parameter while the second impedance is different from the first impedance.

As described above, the embodiments of the invention may be embodied in the form of computer-implemented processes and apparatuses for practicing those processes. Embodiments of the invention may also be embodied in the form of machine-readable medium containing instructions embodied in tangible media, such as floppy diskettes, CD-ROMs, hard drives, or any other machine-readable medium, wherein, when the instructions are loaded into and executed by a computer, the computer becomes an apparatus for practicing the invention.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Moreover, the use of the terms first, second, etc. do not denote any order or importance, but rather the terms first, second, etc. are used to distinguish one element from another.

What is claimed is:

1. A method for providing electrical stimulation therapy to treat pain, comprising:
receiving a first information;
mapping the first information to a first parameter;
generating a first notification signal in accordance with the first parameter;
transmitting the first notification signal;
receiving a second information subsequent to the transmission of the first notification signal;
mapping the second information to a second parameter;
generating a second notification signal in accordance with the second parameter; and
transmitting the second notification signal.

2. The method according to claim 1, wherein the first parameter comprises one or more of an area of interest, a frequency of an electrical signal, a waveform of the electrical signal, a period/time duration of the electrical signal or a magnitude of the electrical signal.

3. The method according to claim 1, wherein the first information comprises a first value indicating a pain level.

4. The method according to claim 3, wherein the second information comprises a second value indicating a pain level.

5. The method according to claim 1, wherein the first information comprises a painful area.

6. The method according to claim 4, wherein the second parameter is different from the first parameter while the second value is different from the first value.

7. The method according to claim 4, wherein the second parameter is the same to the first parameter while the second value is equal to the first value.

8. The method according to claim 1, wherein the first information comprises the user's description.

9. The method according to claim 1, wherein the first information comprises a first skin-impedance.

10. The method according to claim 9, wherein the second information comprises a second skin-impedance.

11. The method according to claim 10, wherein the second parameter is different from the first parameter while the second skin-impedance is different from the first skin-impedance.

12. An apparatus for providing electrical stimulation therapy to treat pain, comprising:
a storage unit/database comprising a first parameter;
a communication module for receiving a first information;
a mapping module for mapping the first information to a first parameter; and
a signal generator for generating a first notification signal in accordance with the first parameter,
wherein the communication module transmits the first notification signal and receives a second information subsequent to the transmission of the first notification signal;
wherein the mapping module maps the second information to a second parameter;
wherein the signal generator generates a second notification signal in accordance with the second parameter; and
wherein the communication module transmits the second notification signal.

13. The apparatus according to claim 12, wherein the communication module receives the first information comprising a first value indicating a pain level.

14. The apparatus according to claim 13, wherein the communication module receives the second information comprising a second value indicating a pain level.

15. The apparatus according to claim 12, wherein the communication module receives the first information comprising a painful area.

16. The apparatus according to claim 14, wherein the communication module receives the second notification signal different from the first notification signal while the second value is different from the first value.

17. The apparatus according to claim 14, wherein the communication module receives the second notification signal equal to the first notification signal while the second value is equal to the first value.

18. The apparatus according to claim 12, wherein the communication module receives the first information comprising the user's description.

19. The apparatus according to claim 12, wherein the communication module receives the skin-impedance of a user.

20. A machine-readable medium including instructions, which when executed by a machine, cause the machine to perform any of the methods of claims 1-11.
